(19) United States
(12) Patent Application Publication
(10) Pub. No.: US 2017/0216296 A1
(43) Pub. Date: Aug. 3, 2017

(54) METHODS OF TREATING HYPERGLYCEMIA

(71) Applicant: Concert Pharmaceuticals, Inc., Lexington, MA (US)

(72) Inventor: LuAnn A. Sabounjian, Holliston, MA (US)

(21) Appl. No.: 15/304,991

(22) PCT Filed: Apr. 15, 2015
(86) PCT No.: PCT/US15/25913

§ 371 Date: Oct. 18, 2016

Related U.S. Application Data
(60) Provisional application No. 61/981,319, filed on Apr. 18, 2014.

(51) Int. Cl. A61K 31/522

(52) U.S. Cl. CPC A61K 31/522

(57) ABSTRACT

The present invention relates to a novel method of treating hyperglycemia in a patient with poor glycemic control. The method comprises administering to the patient an effective amount of a compound described herein.

### CROSS-REFERENCE TO RELATED APPLICATIONS

This application claims the benefit of and priority to U.S. Provisional Patent Application No. 61/981,319, filed Apr. 18, 2014, the contents of which are incorporated herein by reference.

### BACKGROUND OF THE INVENTION

Hyperglycemia, or high blood sugar, is a condition in which an excessive amount of glucose circulates in the blood plasma. Hyperglycemia is most commonly caused by diabetes mellitus. Diabetes mellitus is a serious metabolic disease afflicting over 100 million people worldwide. In the United States, there are more than 12 million diabetics, with 600,000 new cases diagnosed each year. It is increasingly prevalent and results in a high frequency of complications which lead to a significant reduction of life quality and expectancy. Because of diabetes-associated microvascular complications, type 2 diabetes is currently the most frequent cause of adult-onset loss of vision, renal failure, and amputations in the industrialized world. In addition, the presence of type 2 diabetes is associated with a two to five fold increase in cardiovascular disease risk. Diabetes is also a leading cause of damage to the retina at the back of the eye and increases risk of cataracts and glaucoma. Diabetes has also been implicated in the development of kidney disease and with nerve damage, especially in the legs and feet, which interferes with the ability to sense pain and contributes to serious infections. Taken together, diabetes-associated complications are among the leading causes of morbidity and mortality worldwide.

“Good glycemic control” is characterized by maintaining blood glucose levels within acceptable limits and by minimizing fluctuations outside of normal limits between treatments. Persistent poor glycemic control increases the risk of long-term vascular complications of diabetes such as coronary disease, heart attack, stroke, heart failure, kidney failure, blindness, erectile dysfunction, neuropathy (loss of sensation, especially in the feet), gangrene, and gastroparesis (slowed emptying of the stomach). Poor glycemic control also increases the risk of short-term complications of surgery such as poor wound healing.

The AlC test (also known as HbAlC, glycated hemoglobin or glycosylated hemoglobin) is a generally accepted lab test that reflects the average level of blood glucose concentrations over the previous three months. Glycated hemoglobin is formed in a non-enzymatic glycation pathway by hemoglobin's exposure to plasma glucose. Thus, it is not influenced by daily fluctuations in blood glucose levels and serves as a marker for average blood glucose levels over the previous months prior to the measurement. The lab test is used to show how well diabetes is being controlled. Normal levels of HbAlc are less than 5.7%. An HbAlc level of between 5.7% to 6.4% is considered pre-diabetic and a reading of 6.5% or greater is a diagnostic indication of diabetes.

Traditional therapeutic drugs for the treatment of type-2 diabetes, such as metformin, sulfonylureas or insulin, do not always provide good glycemic control. Newer drugs such as the dipeptidyl peptidase 4 inhibitor (DPP-4) class exemplified by sitagliptin, alogliptin and saxagliptin and the thiazolidinedione class exemplified by pioglitazone and rosiglitazone, which bind to peroxisome proliferator-activated receptors (PPARs) are not always tolerated. Although these therapeutic drugs can bring blood glucose levels within normal limits in many patients with hyperglycemia, there are many patients with poor glycemic control who are in need of more effective treatment. Therefore, there is an unmet medical need for new therapeutic agents, especially agents with new mechanisms of action, that are safe and effective for improving glycemic control in patients with hyperglycemia who are not well-regulated by existing medications.

The compound designated herein as “Compound (V) is an oral multi-subtype selective inhibitor of phosphodiesterase enzymes that is described in U.S. Pat. No. 8,263,601 and U.S. Published Application No. 20090239886. It is being developed as an additive treatment to the current standard of care for type 2 diabetic kidney disease, angiotensin modulation, which is treatment with an angiotensin converting enzyme inhibitor or ACEi or an angiotensin receptor blocker or ARB. The compound designated herein as “Compound (V)” is a deuterated analog of 1-(S)-5-hydroxyhexyl-3,7-dimethylxanthine, or HDX, an active metabolite of pentoxifylline.

Pentoxifylline, which forms substantial amounts of HDX, has also been studied in patients with diabetic kidney disease. In a study of 100 patients with type 2 diabetes, pentoxifylline was reported to have no effect on HbAlC. See Ghorbani et al., Nefrología 2012 32(6):790-796.

### SUMMARY OF THE INVENTION

It has now been found that the compounds described herein can substantially reduce the level of glycated hemoglobin (HbAlc) in patients with hyperglycemia, thereby improving glycemic control. During a 48 week clinical trial, patients who were administered a compound of this invention had lower HbAlc relative to patients who received placebo. The patients in this study were type 2 diabetics and were generally already being treated with antihyperglycemic medicines.

This invention is thus directed to a method of treating hyperglycemia in a patient with elevated HbAlc comprising administering to the patient an effective amount of a compound represented by Structural Formula (I):

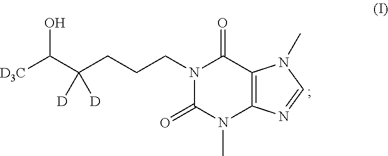 [SMILES: [2H]C([2H])([2H])C(O)C([2H])([2H])CCCn1c(=O)c2c(ncn2C)n(C)c1=O]

or a pharmaceutically acceptable salt thereof. One or more hydrogen atoms in the compound of Structural Formula (I) are optionally substituted with deuterium.

The present invention is also directed to a method of treating hyperglycemia in a patient with poor glycemic control comprising the steps of:

(a) assessing the patient's HbAlc level; and

(b) if the patient's HbAlc level is greater than 5.7% or determined by a physician to be at unacceptably high, administering to the patient an effective amount of a compound of structural formula (I) or a pharmaceutically acceptable salt thereof.

### BRIEF DESCRIPTION OF THE FIGURES

FIG. 1 is a graph showing the LSMean (SE) HbAlC values over weeks for placebo and the treatment arm in the clinical trial described below.

FIG. 2 consists of two bar graphs showing the week 12 HbAlC change from baseline for each patient in the treatment arm and each patient in the placebo arm in the clinical trial described below.

### DETAILED DESCRIPTION OF THE INVENTION

The present invention provides a method of reducing the level of HbAlc in a patient with poor glycemic control. The invention also provides a method of treating hyperglycemia in a patient with poor glycemic control. These methods comprise the step of administering an effective amount of a compound of structural formula (I) or a pharmaceutically acceptable salt thereof to the patient.

“Poor glycemic control” means that the HbAlc level in the patient being treated with the present methods is greater than 5.7%. Alternatively, the HbAlc level is greater than 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7% or 16.8%. In one embodiment, the HbAlc level is greater than 5.9%. Alternatively, the HbAlc level is greater than 6.5%. In another alternative, the HbAlc level is greater than 7.0%.

The invention also provides a method of treating hyperglycemia in a patient with poor glycemic control comprising the steps of: (a) assessing the patient's HbAlc level; and (b) if the patient's HbAlc level is greater than a threshold level, administering to the patient an effective amount of a compound of structural formula (I) or a pharmaceutically acceptable salt thereof. For those patients in whom the HbAlc level is equal to or less than the threshold level, the patient is typically administered an effective amount of a hyperglycemic therapy that does not include the compound of structural formula (I) or a pharmaceutically acceptable salt thereof. Optionally, the method described above further comprises periodically assessing the HbAlc level in the patient and terminating the administration of the compound of formula (I) if the HbAlc level falls below the threshold level. The patient can be periodically assessed at least every month, every two months, every three months, every four months, every five months, every six months, every nine months or every year.

The “threshold level” is the level above which a patient has poor glycemic control, as defined above.

“Hyperglycemic therapy” refers to treatment methods and/or therapeutic agents that are known to be useful in treating hyperglycemia, e.g., in the treatment of diabetes or other metabolic disorder. Examples of hyperglycemic therapies are provided below.

Diseases and disorders causing hyperglycemia and which can be treated by the disclosed methods described are typically metabolic diseases or disorders. Examples include, but are not limited to, Type 1 diabetes (or insulin-dependent diabetes), Type 2 diabetes (or non-insulin dependent diabetes), inadequate glucose tolerance, insulin resistance, hyperlipidemia, hypercholesterolemia, dyslipidemia, syndrome X, metabolic syndrome, obesity, hypertension or atherosclerosis. Also included are drug induced hyperglycemia, such as that resulting from the chronic use of corticosteroids, Zyprexa, Octreotide, and the like; and endocrine disorders of the thyroid, adrenal and pituitary gland, such as Cushing's disease and pancreatitis.

In another embodiment, the patient being treated by the methods described above is also suffering from a diabetes-related disease or condition. A “diabetes related condition” is a disease or disorder that commonly results in patients with diabetes or as consequence of diabetes. One common diabetes related condition treatable with the disclosed methods is.

Other diabetes related conditions may be selected from insulin resistance, retinopathy, diabetic ulcers, acute kidney failure, drug-induced nephrotoxicity, chronic systemic inflammation, neuropathy, nephropathy, atherosclerosis, endothelial dysfunction, osteroporosis, hyperuricemia, gout and hypercoagulability.

In one embodiment, the patient being treated by the methods described above is suffering from type II diabetes.

In another embodiment, the patient being treated by the methods described above is also suffering from chronic kidney disease. In one embodiment, the patient has microalbuminuria. Alternatively, the patient has macroalbuminuria. In another embodiment, the patient has been previously treated with an angiotensin-converting enzyme inhibitor or an angiotensin receptor blocker. Angiotensin-converting enzyme inhibitors and angiotensin receptor blockers are described below. Angiotensin-converting enzyme inhibitor and angiotensin receptor blockers are commonly used in the treatment of kidney disease resulting from diabetes, particularly in hypertensive patients.

As used herein, “microalbuminuria,” refers to conditions whereby albumin is excreted in a patient's urine ranges from 30 mg-300 mg in a 24-hour urine collection or, alternatively, as a concentration of 30-300 mg albumin/liter urine based on a single urine collection. Alternatively, microalbuminuria can refer to the ratio of urinary albumin to creatinine ratio (UACR) measured by spot sampling and/or first morning urine collection. Albumin is normally found in the blood and prevented by the kidney epithelium from passing into urine. When the kidneys are healthy, albumin is generally not present in the urine. But when the epithelium is damaged, albumin can leak into the urine. Microalbuminuria can be diagnosed by standard tests known in the art. In one embodiment, patients with microalbuminuria have between 30 mg-300 mg of albumin in urine over a 24-hour collection period. In another embodiment, microalbuminuria can be diagnosed as a urinary albumin concentration of between 30 mg-300 mg of albumin per liter of urine. Alternatively, patients with microalbuminuria have a ratio of between 30-300 mg albumin/g creatinine (ACR) in a spot sample.

“Macroalbuminuria” refers to conditions whereby albumin excreted in a patient's urine is greater than about 300 mg in a 24 hour period; is greater than 300 mg/L in a spot sample or is greater than 300 mg albumin/g creatinine.

The term “compound,” when referring to a compound used in the disclosed methods, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The term “isotopologue” refers to a species in which the chemical structure differs from a specific compound of this invention only in the isotopic composition thereof. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound. In the compounds of this invention unless otherwise specified any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

It will be recognized that some variation of natural isotopic abundance occurs in a synthesized compound depending upon the origin of chemical materials used in the synthesis. The concentration of naturally abundant stable hydrogen isotopes, notwithstanding this variation, is small and immaterial as compared to the degree of stable isotopic substitution of compounds of this invention. See, for instance, Wada, E et al., Seikagaku, 1994, 66:15; Gannes, L Z et al., Comp Biochem Physiol Mol Integr Physiol, 1998, 119:725.

The term “isotopic enrichment factor” as used herein means the ratio between the isotopic abundance and the natural abundance of a specified isotope.

In yet another embodiment, a compound described herein (e.g., compounds of structural formula (I), (II), (III), (IV) or (V) or a pharmaceutically acceptable salt thereof) has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Throughout this specification, a variable may be referred to generally (e.g., “each Z”) or may be referred to specifically (e.g., Z³, Z⁴, Z⁵, etc.). Unless otherwise indicated, when a variable is referred to generally, it is meant to include all specific embodiments of that particular variable (for example, “Z” includes Z³, Z⁴ and Z⁵).

In a 1^{st} specific embodiment, for the methods described above, the compound of structural formula (I) is represented by the following structural formula:

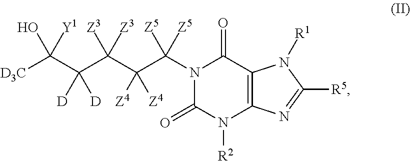

or a pharmaceutically acceptable salt thereof, wherein:
each of R¹ and R² is independently selected from —CH₃ and —CD₃;
R⁵ is hydrogen or deuterium;
each Z³ is hydrogen or deuterium;
each Z⁴ is hydrogen or deuterium;
each Z⁵ is hydrogen or deuterium; and
Y¹ is hydrogen or deuterium.

In one embodiment, for compound of structural formula (II), each Z is hydrogen.

In a 2^{nd} specific embodiment, for methods described above, the compound of structural formula (I) is represented by the following structural formula:

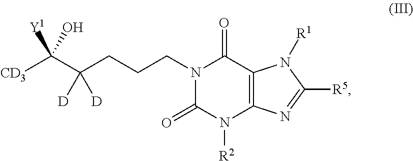

or a pharmaceutically acceptable salt thereof, wherein the variables are as defined above for structural formula (II).

In a 3^{rd} specific embodiment, for methods described in the first and second embodiments, the compound of structural formula (I) is represented by the following structural formula:

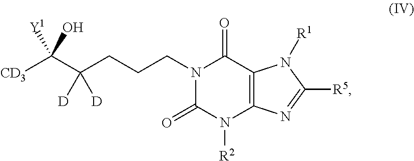

or a pharmaceutically acceptable salt thereof, wherein the variables are as defined above for structural formula (II).

In a 4^{th} specific embodiment, for compounds of structural formula (II), (III) or (IV), R⁵ is deuterium, and the remainder of the variables are as defined in the 1^{st}, 2^{nd} or 3^{rd} specific embodiment.

In a 5^{th} specific embodiment, for compounds of structural formula (II), (III) or (IV), R⁵ is hydrogen, and the remainder of the variables are as defined in the 1^{st}, 2^{nd} or 3^{rd} specific embodiment.

In a 6^{th} specific embodiment, for compounds of structural formula (II), (III) or (IV), R¹ is —CH₃ and R² is —CD₃; and the remainder of the variables are as defined in the 1^{st}, 2^{nd}, 3^{rd} 4^{th} or 5^{th} specific embodiment.

In a 7^{th} specific embodiment, for compounds of structural formula (II), (III) or (IV), R¹ is —CD₃ and R² is —CH₃; and the remainder of the variables are as defined in the 1^{st}, 2^{nd}, 3^{rd} 4^{th} or 5^{th} specific embodiment.

In a 8^{th} specific embodiment, for compounds of structural formula (II), (III) or (IV), R¹ and R² are both —CH₃; and the remainder of the variables are as defined in the 1^{st}, 2^{nd}, 3^{rd} 4^{th} or 5^{th} specific embodiment.

In a 9^{th} specific embodiment, for compounds of structural formula (II), (III) or (IV), R¹ and R² are both —CD₃; and the remainder of the variables are as defined in the 1^{st}, 2^{nd}, 3^{rd}4^{th} or 5^{th} specific embodiment.

In a 10^{th} specific embodiment, for compounds of structural formula (II), (III) or (IV), Y is deuterium; and the remainder of the variables are as defined in the 1^{st}, 2^{nd}, 3^{rd}, 4^{th}, 5^{th}, 6^{th}, 7^{th}, 8^{th} or 9^{th} specific embodiment. Alternatively, Y¹ is hydrogen.

In a 11^{th} specific embodiment, for methods described herein, the compound is represented by the following structural formula:

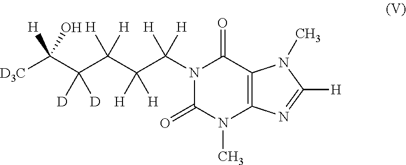 [SMILES: [2H]C([2H])([2H])[C@H](O)C([2H])([2H])CCCn1c(=O)c2c(ncn2C)n(C)c1=O]

or a pharmaceutically acceptable salt thereof.

The invention also provides the use of salts of the compounds described herein.

A salt of a compound of described herein is formed between an acid and a basic group of the compound, such as an amino functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, 3-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The compounds described (e.g., compounds of structural formula (I), (II), (III), (IV) or (V), or a pharmaceutically acceptable salt thereof), contain an asymmetric carbon atom. As such, compounds of this invention can exist as either individual enantiomers, or mixtures of the two enantiomers. Accordingly, a compound used in the disclosed methods may exist as either a racemic mixture or a scalemic mixture, or as individual respective enantiomer that are substantially free from another possible enantiomer. Unless otherwise indicated, when a disclosed compound is named or depicted by a structure without specifying the stereochemistry, it is understood to represent either a racemic mixture or a scalemic mixture, or each individual enantiomer substantially free from the other enantiomer.

When a particular enantiomer of a compound used in the disclosed methods is depicted by name or structure, the enantiomeric purity of the compounds is at least 20%, 30%, 40%, 50%, 60%, 70%, 80%, 90%, 95%, 97%, 99%, 99.5% or 99.9%. “Enantiomeric purity” means the weight percent of the desired enantiomer relative to the combined weight of both enantiomers.

In another embodiment, any atom not designated as deuterium in any of the embodiments set forth above is present at its natural isotopic abundance.

The compounds used in the disclosed methods can be prepared by methods disclosed in U.S. Pat. No. 8,263,601 and U.S. Published Application No. 20090239886, the entire teachings of which are incorporated herein by reference.

The term “mammal” as used herein includes a human or a non-human animal. In one embodiment, the mammal is a non-human animal. In another embodiment, the mammal is a human.

In another embodiment, for methods described herein, the patient is administered an effective amount of a compound described herein. As used herein, the term “effective amount” refers to an amount which, when administered in a proper dosing regimen, is sufficient to therapeutically treat the target disorder. For example, and effective amount is sufficient to reduce or ameliorate the severity, duration or progression of the disorder being treated, slow the advancement of the disorder being treated, cause the regression of the disorder being treated, or enhance or improve the therapeutic effect(s) of another therapy.

The interrelationship of dosages for animals and humans (based on milligrams per meter squared of body surface) is described in Freireich et al., Cancer Chemother. Rep, 1966, 50: 219. Body surface area may be determined approximately from height and weight of the patient. See, e.g., Scientific Tables, Geigy Pharmaceuticals, Ardsley, N.Y., 1970, 537.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the prescribing information for pentoxifylline.

In one embodiment, for methods described herein, the compound described herein (e.g., compounds of formula (I), (II), (III), (IV) or (V), or a pharmaceutically acceptable salt thereof) is administered to the patient at a dosage range of 50 mg/day to 3000 mg/day, 100 mg/day to 2500 mg/day, 200 mg/day to 2000 mg/day, 300 mg/day to 2000 mg/day, 600 mg/day to 1500 mg/day, 650 mg/day to 1500 mg/day or 900 mg/day to 1500 mg/day. In one embodiment, the patient is administered with the compound at a dosage of 200 mg/day, 300 mg/day, 400 mg/day, 600 mg/day, 900 mg/day, 1200 mg/day or 1500 mg/day. In one embodiment, any one of these dosages are administered once per day. Alternatively, any one of these dosages are administered twice per day.

In another embodiment, the compound can be administered to the patient once a day, twice a day, three times a day, four time a day, once every other day, two or three times a week, or every week. In one embodiment, the compound is administered twice a day. In another embodiment, the compound is administered to the patient twice daily with 600 mg each time.

Optionally, any of the above methods of treatment can further comprise co-administering to the patient an effective amount of one or more second therapeutic agents.

Exemplary agents for co-administration in the disclosed methods include those described in WO 1997019686, EP 0640342, WO 2003013568, WO 2001032156, WO 2006035418, and WO 1996005838, the entire teachings of which are incorporated herein by reference.

In one embodiment, the second therapeutic agent is selected from an angiotensin-converting enzyme (ACE) inhibitor and an angiotensin receptor blocker (ARB). Specific examples of ACE inhibitors include, but are not limited to, benazepril (LOTENSIN), captopril (CAPOTEN), enalapril (VASOTEC), fosinopril (MONOPRIL), lisinopril (PRINIVIL, ZESTRIL), moexipril (UNIVASC), perindopril (ACEON), quinapril (ACCUPRIL), ramapril (ALTACE), and trandolapril (MAVIK). Specific examples of ARBs include, but are not limited to, candesartan (ATACAND), eprosartan (TEVETEN), irbesartan (AVAPRO), losartan (COZAAR), olmesartan (BENICAR), telmisartan (MICARDIS) and valsartan (DIOVAN).

In one embodiment, the second therapeutic agent is selected from α-tocopherol and hydroxyurea.

In another embodiment, the second therapeutic agent is used as a hyperglycemic therapy. Examples include insulin or insulin analogues, glucagon-like-peptide-1 (GLP-1) receptor agonists, sulfonylurea agents, biguanide agents, alpha-glucosidase inhibitors, PPAR agonists, meglitinide agents, dipeptidyl-peptidase (DPP) IV inhibitors, other phosphodiesterase (PDE1, PDE2, PDE3, PDE4, PDE5, PDE9, PDE10 or PDE11) inhibitors, amylin agonists, CoEnzyme A inhibitors, and antiobesity agents.

Specific examples of insulin include, but are not limited to Humulin® (human insulin, rDNA origin), Novolin® (human insulin, rDNA origin), Velosulin® BR (human buffered regular insulin, rDNA origin), Exubera® (human insulin, inhaled), and other forms of inhaled insulin, for instance, as delivered by Mannkind's “Technosphere Insulin System”.

Specific examples of insulin analogues include, but are not limited to, novarapid, insulin detemir, insulin lispro, insulin glargine, insulin zinc suspension and Lys-Pro insulin.

Specific examples of Glucagon-Like-Peptide-1 receptor agonists include, but are not limited to BIM-51077 (CAS-No. 275371-94-3), EXENATIDE (CAS-No. 141758-74-9), CJC-1131 (CAS-No. 532951-64-7), LIRAGLUTIDE (CAS-No. 20656-20-2) and ZP-10 (CAS-No. 320367-13-3).

Specific examples of sulfonylurea agents include, but are not limited to, TOLBUTAMIDE (CAS-No. 000064-77-7), TOLAZAMIDE (CAS-No. 001156-19-0), GLIPIZIDE (CAS-No. 029094-61-9), CARBUTAMIDE (CAS-No. 000339-43-5), GLISOXEPIDE (CAS-No. 025046-79-1), GLISENTIDE (CAS-No. 032797-92-5), GLIBORNURIDE (CAS-No. 026944-48-9), GLIBENCLAMIDE (CAS-NO. 010238-21-8), GLIQUIDONE (CAS-No. 033342-05-1), GLIMEPIRIDE (CAS-No. 093479-97-1) and GLICLAZIDE (CAS-No. 021187-98-4).

A specific example of a biguanide agent includes, but is not limited to METFORMIN (CAS-No. 000657-24-9).

Specific examples of alpha-glucosidase-inhibitors include, but are not limited to ACARBOSE (Cas-No. 056180-94-0), MIGLITOL (CAS-No. 072432-03-2) and VOGLIBOSE (CAS-No. 083480-29-9).

Specific examples of PPAR-agonists include, but are not limited to MURAGLITAZAR (CAS-No. 331741-94-7), ROSIGLITAZONE (CAS-NO. 122320-73-4), PIOGLITAZONE (CAS-No. 111025-46-8), RAGAGLITAZAR (CAS-NO. 222834-30-2), FARGLITAZAR (CAS-No. 196808-45-4), TESAGLITAZAR (CAS-No. 251565-85-2), NAVEGLITAZAR (CAS-No. 476436-68-7), NETOGLITAZONE (CAS-NO. 161600-01-7), RIVOGLITAZONE (CAS-NO. 185428-18-6), K-1 11 (CAS-No. 221564-97-2), GW-677954 (CAS-No. 622402-24-8), FK-614 (CAS-No 193012-35-0) and (-)-Halofenate (CAS-No. 024136-23-0). Preferred PPAR-agonists are ROSGLITAZONE and PIOGLITAZONE.

Specific examples of meglitinide agents include, but are not limited to REPAGLINIDE (CAS-No. 135062-02-1), NATEGLINIDE (CAS-No. 105816-04-4) and MITIGLINIDE (CAS-No. 145375-43-5).

Specific examples of DPP IV inhibitors include, but are not limited to SITAGLIPTIN (CAS-No. 486460-32-6), SAXAGLIPTIN (CAS-No. 361442-04-8), VILDAGLIPTIN (CAS-No. 274901-16-5), DENAGLIPTIN (CAS-No. 483369-58-0), ALOGLIPTIN (CAS-No. 850649-62-6), P32/98 (CAS-No. 251572-70-0) and NVP-DPP-728 (CAS-No. 247016-69-9).

Specific examples of PDE5 inhibitors include, but are not limited to SILDENAFIL (CAS-No. 139755-83-2), VARDENAFIL (CAS-No. 224785-90-4) and TADALAFIL (CAS-No. 171596-29-5). Examples of PDE1, PDE9, PDE10 or PDE11 inhibitors which may be usefully employed according to the present invention can be found, for example, in US20020160939, WO2003037432, US2004220186, WO2005/003129, WO2005012485, WO2005120514 and WO03077949.

A specific example of an amylin agonist includes, but is not limited to PRAMLINITIDE (CAS-No. 151126-32-8).

A specific example of a Coenzyme A inhibitor includes, but is not limited to ETOMOXIR (CAS-No. 082258-36-4).

Specific examples of anti-obesity drugs include, but are not limited to HMR-1426 (CAS-No. 262376-75-0), CETILISTAT (CAS-No. 282526-98-1) and SIBUTRAMINE (CAS-No. 106650-56-0).

In particular, the combination therapies of this invention include co-administering an effective amount of a compound described herein (e.g., a compound of structural formula (I), (II), (III), (IV), (V) or a pharmaceutically acceptable salt thereof) and an effective amount of a second therapeutic agent for treatment of the following conditions (with the particular second therapeutic agent indicated in parentheses following the indication): late radiation induced injuries (α-tocopherol), radiation-induced fibrosis (α-tocopherol), radiation induced lymphedema (α-tocopherol), chronic breast pain in breast cancer patients (α-tocopherol), type 2 diabetic nephropathy (captopril), malnutrition-inflammation-cachexia syndrome (oral nutritional supplement, such as Nepro; and oral anti-inflammatory module, such as Oxepa); and brain and central nervous system tumors (radiation therapy and hydroxyurea).

The term “co-administered” as used herein means that the second therapeutic agent may be administered together with a compound described herein (e.g., a compound of structural formula (I), (II), (III), (IV), (V) or a pharmaceutically acceptable salt thereof) as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound described herein. In such combination therapy treatment, both the compounds used in the disclosed methods and the second therapeutic agent(s) are administered by conventional methods. The administration of a combination used in the disclosed methods, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In one embodiment, for methods described herein, a composition comprising an effective amount of a compound described herein (e.g., compounds of structural formula (I), (II), (III), (IV), (V) or a pharmaceutically acceptable salt thereof) can be used. In one embodiment, the composition is a pharmaceutical compositions comprising an effective amount of a compound described herein (e.g., compounds of structural formula (I), (II), (III), (IV), (V) or a pharmaceutically acceptable salt thereof) and an acceptable carrier.

In one embodiment, the pharmaceutical composition is pyrogen-free. Preferably, a composition of this invention is formulated for pharmaceutical use (“a pharmaceutical composition”), wherein the carrier is a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds described herein in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound described herein optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

In another embodiment, a composition described above further comprises a second therapeutic agent. In another embodiment, the compound described herein and one or more of any of the above-described second therapeutic agents are in separate dosage forms, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

### Clinical Trial

A Phase 2 placebo-controlled clinical trial of Compound (V) was conducted in patients with type 2 diabetic kidney disease and macroalbuminuria. The purpose of the trial was to compare Compound (V) to placebo in terms of safety and efficacy with respect to measures of renal function such as urinary albumin to creatinine ratio, serum creatinine levels, and estimated glomerular filtration rate. HbAlc was measured but was not an intended endpoint of the trial. Thus, while the study was controlled for other medications, it was not controlled for anti-glycemic medications nor were the placebo and treated groups matched for baseline HbAlc levels.

All patients enrolled in the clinical trial were concurrently treated with angiotensin modulators. The clinical trial consists of three parts, two of which were completed and a third part that is on-going. Part 1 was a double-blind, parallel, two-arm, placebo-controlled study evaluating the safety and efficacy of 600 mg of Compound (V) twice daily for 24 weeks. 182 patients were enrolled in this first part of the trial. Part 2 was a blinded 24-week extension study in which all patients who completed Part 1 were eligible to continue receiving 600 mg of Compound (V) or placebo twice daily. 143 patients were enrolled in this part of the clinical trial and have completed dosing. 123 of the 143 patients that were enrolled in Part 2 of the clinical trial completed Part 2. The combined 48 weeks of data from Parts 1 and 2 of the clinical trial have been analyzed. 102 patients enrolled in Part 3 which is a 48 week open-label extension study that is on-going. All patients who completed Part 2 were eligible to receive 600 mg of Compound (V) twice daily.

The key criteria for patients to be included in the clinical trial were (a) eGFR from 23 to 89 mL/min/1.73 m2, a measure of kidney function which indicates mild to moderately severe type 2 diabetic kidney disease; (b) having been on a stable angiotensin modulation regimen for a minimum of four weeks prior to initiating screening and nine weeks prior to initiating dosing; (c) blood pressure less than or equal to 145/90 mm Hg; (d) glycosylated hemoglobin Alc (HbAlc) less than or equal to 10.5%; and (e) UACR greater than or equal to 200 mg/g in male patients and 300 mg/g in female patients, ratios of albumin to creatinine that are indicative of substantial kidney damage in men and women, but not more than 5,000 mg/g, a ratio indicative of severe kidney disease. HbAlc in whole blood samples was measured by ion-exchange chromatography using a TOSOH HLC-723G8 HPLC analyzer.

The procedure is similar to the one described in the Laboratory Procedure Manual of the Centers for Disease Control and Prevention. See www.cdc.gov/nchs/data/nhanes/nhanes_11_12/GHB_met_G_Tosoh_G8.pdf. The TOSOH instrument can reliably detect HbAlc levels in the range of 4.0-16.8%.

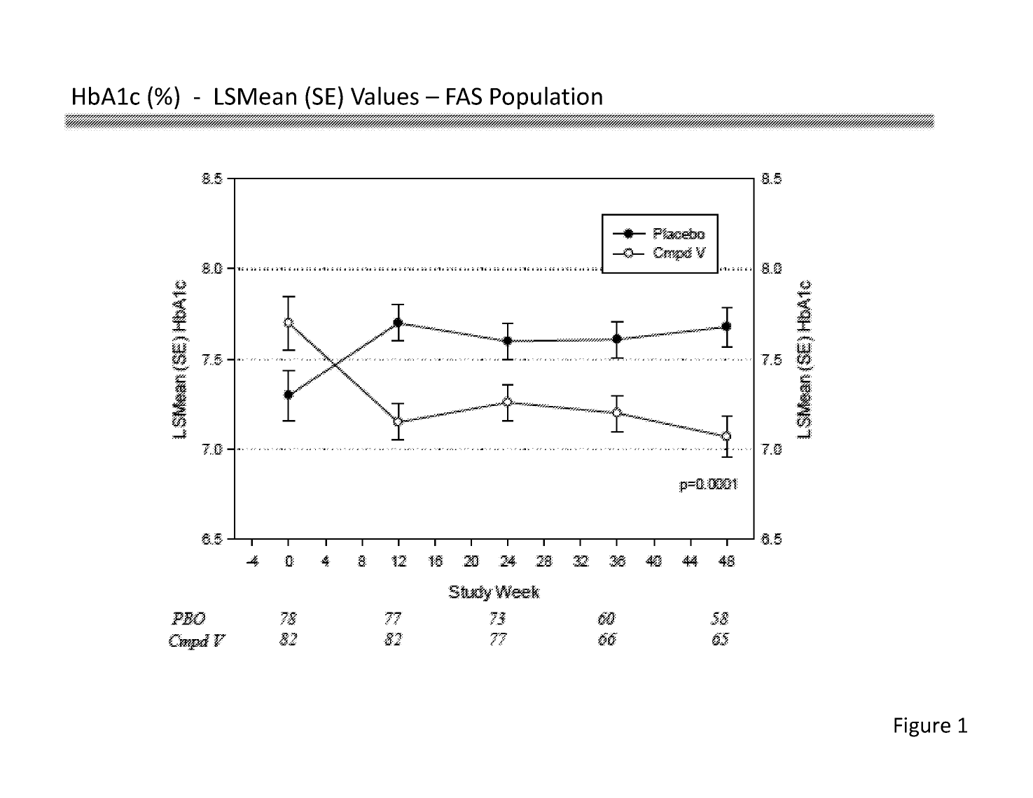

Even though HbAlc was not an intended primary or secondary endpoint in this clinical trial, an unanticipated positive effect on hemoglobin glycosylation was observed in the group receiving Compound (V). Patients were sampled at weeks 0, 12, 24, 36 and 48 weeks (see FIG. 1). The value at 0 weeks was calculated as the mean of values recorded on Days −28, −7 and −1. In other words, the patients were tested 4 weeks before the trial, one week before the trial and one day before. The mean value of the measurements was recorded as Day 0 for the trial.

FIG. 1 shows the preliminary LSMean (SE) HbAlc levels of the patient population over the course of the trial. At the beginning of the trial the group treated with Compound (V) had a least square mean HbAlc value of 7.7 which dropped to 7.1 during Part 1 and Part 2 of the trial. In the placebo group, the HbAlc levels rose from 7.3 to 7.4. The lowering of HbAlc in the treated group relative to placebo was statistically significant (p=0.0001).

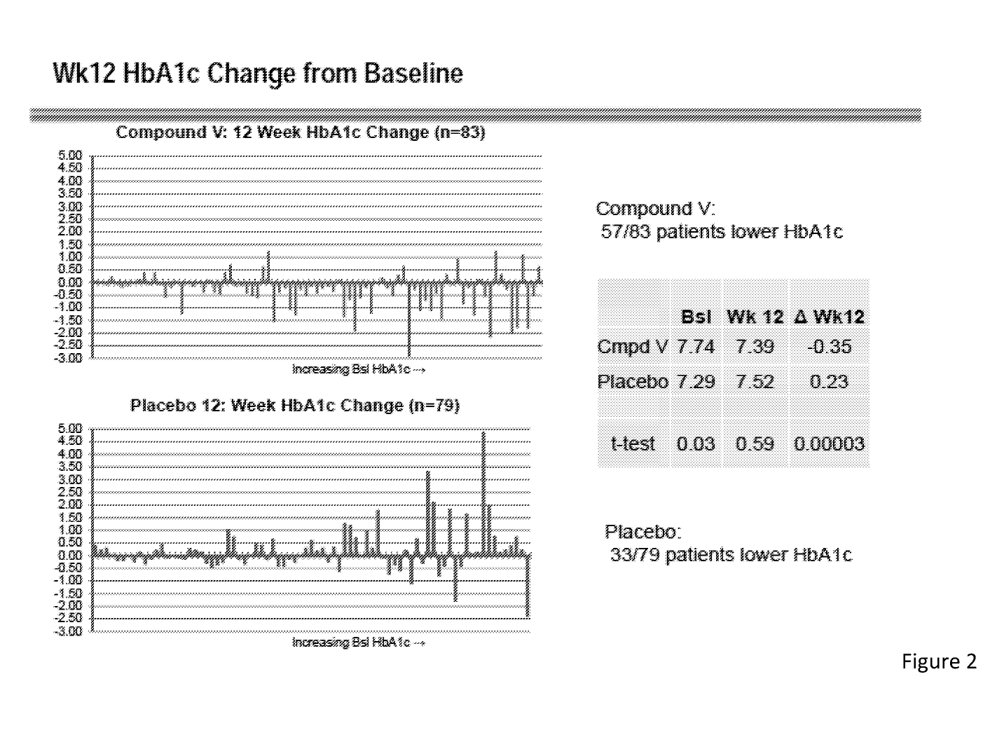

FIG. 2 shows the HbAlc change from baseline for each patient in the trial. 57/83 patients on Compound V had lower HbAlc after 12 weeks versus 33/79 patients on placebo. Overall, the Compound V group experienced a 0.35 drop in AlC versus a 0.23 increase in the placebo group that was statistically significant.

What is claimed is:

1. A method of treating hyperglycemia in a patient with poor glycemic control comprising administering to the patient an effective amount of a compound represented by the following structural formula: 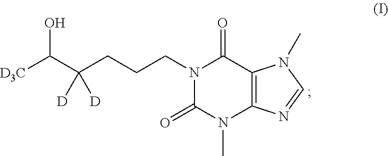 [SMILES: [2H]C([2H])([2H])C(O)C([2H])([2H])CCCn1c(=O)c2c(ncn2C)n(C)c1=O]
or a pharmaceutically acceptable salt thereof, wherein one or more hydrogen atoms are optionally substituted with deuterium.

2. The method of claim 1, wherein the poor glycemic control is an HbAlc level that is greater than 5.7% before the treatment.

3. The method of claim 1, wherein the poor glycemic control is an HbAlc level that is greater than 6.5% before the treatment.

4. The method of claim 1, wherein the poor glycemic control is an HbAlc level that is greater than 7.0% before the treatment.

5. A method of treating hyperglycemia in a patient with poor glycemic control comprising the steps of:
(a) assessing the patient's HbAlc level; and
(b) if the patient's HbAlc level is greater than 5.7%, administering to the patient an effective amount of a compound represented by the following structural formula: 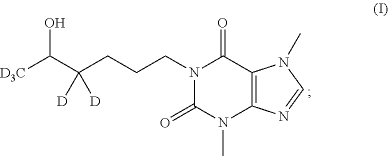 [SMILES: [2H]C([2H])([2H])C(O)C([2H])([2H])CCCn1c(=O)c2c(ncn2C)n(C)c1=O]
or a pharmaceutically acceptable salt thereof, wherein one or more hydrogen atoms are optionally substituted with deuterium.

6. The method of claim 5, wherein the patient's HbAlc level is greater than 6.5%.

7. The method of claim 5, wherein the patient's HbAlc level is greater than 7.0%.

8. The method of claim 1, wherein the compound is administered at a dosage range of 300 mg/day to 2400 mg/day.

9. The method of claim 8, wherein the compound is administered at a dosage range of 600 mg/day to 1200 mg/day.

10. The method of claim 8, wherein the compound is administered at a dosage level of 600 mg/day, 900 mg/day or 1200 mg/day.

11. (canceled)

12. The method of claim 1, wherein the compound is represented by the following structural formula: 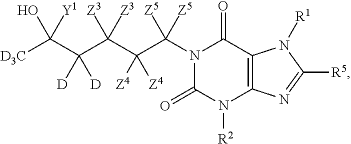
or a pharmaceutically acceptable salt thereof, wherein:
each of R¹ and R² is independently selected from —CH₃ and —CD₃;
R⁵ is hydrogen or deuterium;
each Z³ is hydrogen or deuterium;
each Z⁴ is hydrogen or deuterium;
each Z⁵ is hydrogen or deuterium; and
Y¹ is hydrogen or deuterium.

13. The method of claim 12, wherein each Z is hydrogen.

14. The method of claim 13, wherein the compound is represented by the following structural formula: 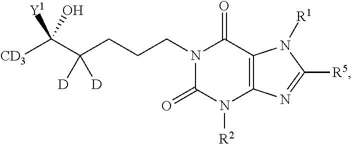
or a pharmaceutically acceptable salt thereof.

15. The method of claim 13, wherein the compound is represented by the following structural formula: 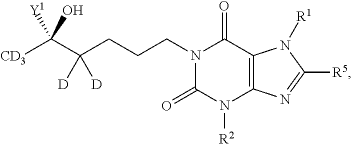
or a pharmaceutically acceptable salt thereof.

16.-19. (canceled)

20. The method of claim 12, wherein R¹ and R² are both —CH₃.

21.-23. (canceled)

24. The method of claim 12, wherein the compound is 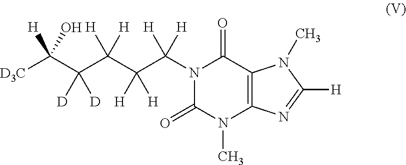 [SMILES: [2H]C([2H])([2H])[C@H](O)C([2H])([2H])CCCn1c(=O)c2c(ncn2C)n(C)c1=O]
or a pharmaceutically acceptable salt thereof.

25. The method of claim 14, wherein the enantiomeric purity of the compound is at least 90%.

26. The method of claim 14, wherein the enantiomeric purity of the compound is at least 95%.

27. The method of claim 14, wherein the enantiomeric purity of the compound is at least 99%.

28. The method of claim 12, wherein any hydrogen atom not designated as deuterium is present at its natural isotopic abundance.

29. (canceled)

30. (canceled)
